(19) United States
(12) Patent Application Publication
(10) Pub. No.: US 2023/0391850 A1
(43) Pub. Date: Dec. 7, 2023

(54) Separation Matrix

(71) Applicant: Cytiva BioProcess R&D AB, Uppsala (SE)

(72) Inventors: Mats Ander, Uppsala (SE); Gustav Rodrigo, Uppsala (SE); Tomas Björkman, Uppsala (SE)

(21) Appl. No.: 18/249,525

(22) PCT Filed: Nov. 10, 2021
(86) PCT No.: PCT/EP2021/081214

§ 371 Date: Apr. 18, 2023

(30) Foreign Application Priority Data
Nov. 26, 2020 (GB) 2018588.0

(51) Int. Cl. C07K 16/06; B01D 15/38; B01J 20/286; B01J 20/32; B01J 20/28; C07K 14/31

(52) U.S. Cl. CPC C07K 16/065; B01D 15/3809; B01J 20/286; B01J 20/3212; B01J 20/3274; B01J 20/28038; C07K 14/31

(57) ABSTRACT

The invention relates to an alkali-stable mutated Fc-binding Protein A domain, having at least 95% identity to SEQ ID NO: 8 or SEQ ED NO: 9.

### TECHNICAL FIELD OF THE INVENTION

The present invention relates to the field of affinity chromatography, and more specifically to mutated immunoglobulin-binding domains of Protein A, which are useful in affinity chromatography of immunoglobulins. The invention also relates to multimers of the mutated domains and to separation matrices containing the mutated domains or multimers.

### BACKGROUND OF THE INVENTION

Immunoglobulins represent the most prevalent biopharmaceutical products in either manufacture or development worldwide. The high commercial demand for and hence value of this particular therapeutic market has led to the emphasis being placed on pharmaceutical companies to maximize the productivity of their respective mAb manufacturing processes whilst controlling the associated costs.

Affinity chromatography is used in most cases, as one of the key steps in the purification of these immunoglobulin molecules, such as monoclonal or polyclonal antibodies. A particularly interesting class of affinity reagents is proteins capable of specific binding to invariable parts of an immunoglobulin molecule, such interaction being independent on the antigen-binding specificity of the antibody. Such reagents can be widely used for affinity chromatography recovery of immunoglobulins from different samples such as but not limited to serum or plasma preparations or cell culture derived feed stocks. An example of such a protein is staphylococcal protein A, containing domains capable of binding to the Fc and also Fab (via the V_{H}3 domain) portions of IgG immunoglobulins from different species. These domains are commonly denoted as the E-, D-, A-, B- and C-domains (SEQ ID NO: 1-5).

Staphylococcal protein A (SpA) based reagents have due to their high affinity and selectivity found a widespread use in the field of biotechnology, e.g. in affinity chromatography for capture and purification of antibodies as well as for detection or quantification. At present, SpA-based affinity medium probably is the most widely used affinity medium for isolation of monoclonal antibodies and their fragments from different samples including industrial cell culture supernatants. Accordingly, various matrices comprising protein A-ligands are commercially available, for example, in the form of native protein A (e.g. Protein A SEPHAROSE™, Cytiva, Uppsala, Sweden) and also comprised of recombinant protein A (e.g. rProtein A-SEPHAROSE™, Cytiva). More specifically, the genetic manipulation performed in the commercial recombinant protein A product is aimed at facilitating the attachment thereof to a support and at increasing the productivity of the ligand.

These applications, like other affinity chromatography applications, require comprehensive attention to definite removal of contaminants. Such contaminants can for example be non-eluted molecules adsorbed to the stationary phase or matrix in a chromatographic procedure, such as non-desired biomolecules or microorganisms, including for example proteins, carbohydrates, lipids, bacteria and viruses. The removal of such contaminants from the matrix is usually performed after a first elution of the desired product, in order to regenerate the matrix before subsequent use. Such removal usually involves a procedure known as cleaning-in-place (CIP), wherein agents capable of eluting contaminants from the stationary phase are used. One such class of agents often used is alkaline solutions that are passed over said stationary phase. At present the most extensively used cleaning and sanitizing agent is NaOH, and the concentration thereof can range from 0.1 up to e.g. 1 M, depending on the degree and nature of contamination. This strategy is associated with exposing the matrix to solutions with pH-values above 13. For many affinity chromatography matrices containing proteinaceous affinity ligands such alkaline environment is a very harsh condition and consequently results in decreased capacities owing to instability of the ligand to the high pH involved.

An extensive research has therefore been focused on the development of engineered protein ligands that exhibit an improved capacity to withstand alkaline pH-values. For example, Güllich et al. (Susanne Gülick, Martin Linhult, Per-Åke Nygren, Mathias Uhlén, Sophia Hober, Journal of Biotechnology 80 (2000), 169-178) suggested protein engineering to improve the stability properties of a Streptococcal albumin-binding domain (ABD) in alkaline environments. Güilich et al. created a mutant of ABD, wherein all the four asparagine residues have been replaced by leucine (one residue), aspartate (two residues) and lysine (one residue). Further, Güllich et al. report that their mutant exhibits a target protein binding behavior similar to that of the native protein, and that affinity columns containing the engineered ligand show higher binding capacities after repeated exposure to alkaline conditions than columns prepared using the parental non-engineered ligand. Thus, it is concluded therein that all four asparagine residues can be replaced without any significant effect on structure and function.

Recent work shows that changes can also be made to protein A (SpA) to effect similar properties. U.S. Pat. No. 7,834,158, which is hereby incorporated by reference in its entirety, discloses that when at least one asparagine residue is mutated to an amino acid other than glutamine or aspartic acid, the mutation confers an increased chemical stability at pH-values of up to about 13-14 compared to the parental SpA, such as the B-domain of SpA, or Protein Z (SEQ ID NO: 6), a synthetic construct derived from the B-domain of SpA (U.S. Pat. No. 5,143,844, incorporated by reference in its entirety). The authors show that when these mutated proteins are used as affinity ligands, the separation matrices as expected can better withstand cleaning procedures using alkaline agents. This applies in particular to Protein Z with the mutations N3A,N6D,N23T (SEQ ID NO: 7, herein denoted as Zvar), as disclosed in U.S. Pat. No. 8,198,404, hereby incorporated by reference in its entirety. Further mutations of protein A domains with the purpose of increasing the alkali stability have also been published in US 8,329,860, JP 2006304633A, U.S. Pat. No. 8,674,073, U.S. Pat. No. 10,072,050, U.S. Pat. No. 9,403,883, U.S. Pat. No. 9,051,375 U.S. Pat. No. 9,051,375, U.S. Pat. No. 9,683,013, US 2019/048046 and U.S. Pat. No. 10,703,774 all of which are hereby incorporated by reference in their entireties. However, there is still a need for mutants with higher alkali stability, allowing a higher number of cleaning cycles with NaOH before the separation matrix has to be discarded due to capacity loss.

There is thus still a need in this field to obtain a separation matrix containing protein ligands having a further improved stability towards alkaline cleaning procedures. There is also a need for such separation matrices with an improved binding capacity to allow for economically efficient purification of therapeutic antibodies.

### SUMMARY OF THE INVENTION

One aspect of the invention is to provide a polypeptide with improved alkaline stability. This is achieved with an Fc-binding polypeptide comprising an amino acid sequence as defined by, or having at least 95%, such as at least 98% identity to, SEQ ID NO: 8 or SEQ ID NO: 9. Alternatively, the polypeptide comprises a sequence as defined by, or having at least 98% identity to SEQ ID NO 11.

(SEQ ID NO 11)
X₁Q X₂AFYEILHLP NLTEEQRNAF IQSLKDDPSX₃ SKAILAEAKK
LNDAQ

wherein individually of each other:

### X₁=A or W

### X₂=E or R

### X₃=V or Q,

with the proviso that when X₁ is A, X₂=R and when X₂=E, X₁=W.

One advantage is that the alkaline stability is improved over the parental polypeptides, with a maintained highly selective binding towards immunoglobulins and other Fc-containing proteins.

A second aspect of the invention is to provide a multimer with improved alkaline stability, comprising a plurality of polypeptides. This is achieved with a multimer of the polypeptide disclosed above.

A third aspect of the invention is to provide a nucleic acid or a vector encoding a polypeptide or multimer with improved alkaline stability. This is achieved with a nucleic acid or vector encoding a polypeptide or multimer as disclosed above.

A fourth aspect of the invention is to provide an expression system capable of expressing a polypeptide or multimer with improved alkaline stability. This is achieved with an expression system comprising a nucleic acid or vector as disclosed above.

A fifth aspect of the invention is to provide a separation matrix capable of selectively binding immunoglobulins and other Fc-containing proteins and exhibiting an improved alkaline stability. This is achieved with a separation matrix comprising polypeptides or multimers as described above covalently coupled to a porous support.

One advantage is that a high dynamic binding capacity is provided. A further advantage is that a high degree of alkali stability is achieved.

A sixth aspect of the invention is to provide an efficient and economical method of isolating an immunoglobulin or other Fc-containing protein. This is achieved with a method comprising the steps of:

a) contacting a liquid sample comprising an immunoglobulin with a separation matrix as disclosed above,
b) washing the separation matrix with a washing liquid,
c) eluting the immunoglobulin from the separation matrix with an elution liquid, and
d) cleaning the separation matrix with a cleaning liquid.

Further suitable embodiments of the invention are described in the dependent claims.

### Definitions

The terms “antibody” and “immunoglobulin” are used interchangeably herein, and are understood to include also fragments of antibodies, fusion proteins comprising antibodies or antibody fragments and conjugates comprising antibodies or antibody fragments.

The terms an “Fc-binding polypeptide” and “Fc-binding protein” mean a polypeptide or protein respectively, capable of binding to the crystallisable part (Fc) of an antibody and includes e.g. Protein A and Protein G, or any fragment or fusion protein thereof that has maintained said binding property.

The term “linker” herein means an element linking two polypeptide units, monomers or domains to each other in a multimer.

The term “spacer” herein means an element connecting a polypeptide or a polypeptide multimer to a support.

The term “% identity” with respect to comparisons of amino acid sequences is determined by standard alignment algorithms such as, for example, Basic Local Alignment Tool (BLAST™) described in Altshul et al. (1990) J. Mol. Biol., 215: 403-410. A web-based software for this is freely available from the US National Library of Medicine at http://blast.ncbi.nlm.nih.gov/Blast.cgi?PROGRAM=blastp&PAGE_TYPE=BlastSearch&LINK_LOC=blasthome. Here, the algorithm “blastp (protein-protein BLAST)” is used for alignment of a query sequence with a subject sequence and determining i.a. the % identity.

As used herein, the terms “comprises,” “comprising,” “containing,” “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### BRIEF DESCRIPTION OF FIGURES

FIG. 1 shows an alignment of the Fc-binding domains as defined by SEQ ID NO:1-7.

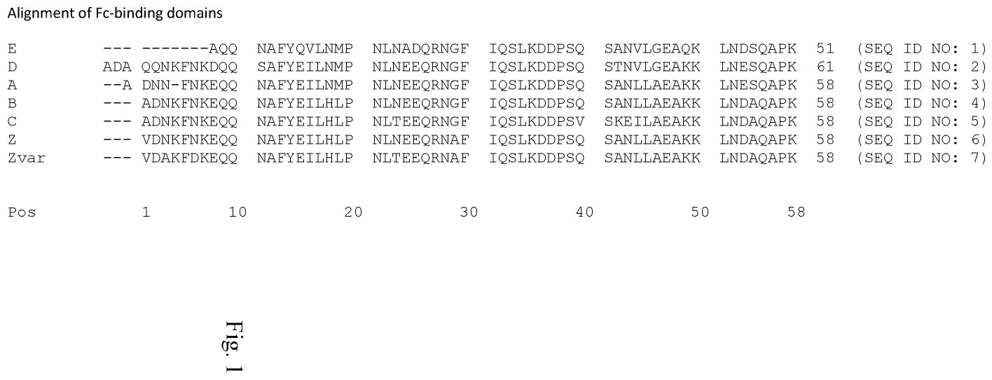

### DETAILED DESCRIPTION OF EMBODIMENTS

In one aspect the present invention discloses an Fc-binding polypeptide, which

comprises an amino acid sequence as defined by, or having at least 95%, such as at least 98% identity to, SEQ ID NO: 8 or SEQ ID NO: 9. Additionally it discloses an Fc-binding polypeptide, which comprises an amino acid sequence as defined by, or having at least 95%, such as at least 98% identity to, SEQ ID NO: 10. The mutations at positions 1 and 3 in these domains confers an improved alkali stability in comparison with the parental domain/polypeptide, without impairing the immunoglobulin-binding properties. Hence, the polypeptide can also be described as an Fc- or immunoglobulin-binding polypeptide, or alternatively as an Fc- or immunoglobulin-binding polypeptide unit. It can further be described as an alkali-stable Fc- or immunoglobulin-binding polypeptide. In addition, the polypeptides are capable of binding to the V_{H}3 domain of the Fab portion of IgG, which means that they can also be used for capture of e.g. V_{H}3-containing Fab fragments. This is in contrast to the Protein Z-derived alkali-stable Protein A resin MabSelect™ SuRe (Cytiva), which does not bind to V_{H}3 (T A Seldon et al: J Biomolecular Techniques 22(2), 2011, 50-52).

SEQ ID NO: 8
AQ RAFYEILHLP NLTEEQRNAF IQSLKDDPSV SKAILAEAKK
LNDAQ
SEQ ID NO: 9
WQ EAFYEILHLP NLTEEQRNAF IQSLKDDPSV SKAILAEAKK
LNDAQ
SEQ ID NO: 10
WQ RAFYEILHLP NLTEEQRNAF IQSLKDDPSV SKAILAEAKK
LNDAQ

The (alkali-stable) Fc- or immunoglobulin-binding polypeptide can also be described as comprising a sequence as defined by, or having at least 98% identity to SEQ ID NO: 11.

(SEQ ID NO 11)
X₁Q X₂AFYEILHLP NLTEEQRNAF IQSLKDDPSX₃ SKAILAEAKK
LNDAQ

wherein individually of each other:

### X₁=A or W

### X₂=E or R

### X₃=V or Q,

with the proviso that when X₁ is A, X ₂=R and when X₂=E, X₁=W.

The polypeptide may further comprise additional amino acid residues at the N- and/or C-terminal end, e.g. a leader sequence at the N-terminal end and/or a tail sequence at the C-terminal end. The leader sequence may e.g. be a 1-20 amino acid sequence, such as a 3-20, 4-20 or 6-8 amino acid sequence. More specifically it can be defined by or have at least 80% identity, such as at least 90% identity or at least 95% identity with an amino acid sequence selected from the group consisting of VFDKE, AKFDKE, VDA, VDAKFDKE, KFDKE, KVDKE, KADKE, ADNKFNKE, VDNKFNKE, YEDGVDAKFDKE, AQYEDGKQYTDT and AQYEDGKQYTDTVDAKFDKE, or alternatively selected from the group consisting of VFDKE, AKFDKE, VDA, VDAKFDKE, KFDKE, KVDKE, KADKE, YEDGVDAKFDKE, AQYEDGKQYTDT and AQYEDGKQYTDTVDAKFDKE. In particular, the leader sequence can be defined by or have at least 80% identity, such as at least 90% identity or at least 95% identity, with the amino acid sequence VDAKFDKE. The tail sequence may e.g. be a 1-5 amino acid sequence, such as a 2-4 amino acid sequence. More specifically it can be defined by or have at least 60% identity with an amino acid sequence selected from the group consisting of AP, APK and APA, such as the amino acid sequence APK. Suitably, the leader and the tail do not contain any asparagine residues. Further, the tail can advantageously comprise a proline. Suitable leader and tail sequences are further described in U.S. Pat. No. 10,703,774, hereby incorporated by reference in its entirety. Accordingly, the polypeptide can have a structure as described below:

### [Leader]-[SEQ ID NO: 8, 9, 10 or 11]-[Tail]

where Leader and Tail are as described above.

In a second aspect the present invention discloses a multimer comprising, or consisting essentially of, a plurality of linked polypeptides as defined by any embodiments disclosed above. The use of multimers may increase the immunoglobulin binding capacity and multimers may also have a higher alkali stability than monomers. The multimer can e.g. be a dimer, a trimer, a tetramer, a pentamer, a hexamer, a heptamer, an octamer or a nonamer. It can be a homomultimer, where all the polypeptides in the multimer are identical or it can be a heteromultimer, where at least one unit differs from the others. Advantageously, all the polypeptides in the multimer are alkali stable, such as by comprising the sequences disclosed above. The polypeptides can be linked to each other directly by peptide bonds between the C-terminal and N-terminal ends of the polypeptides. Alternatively, two or more polypeptides in the multimer can be linked by linkers comprising oligomeric or polymeric species, such as linkers comprising peptides with up to 25 or 30 amino acids, such as 3-25 or 3-20 amino acids. If the polypeptides comprise leader and/or tail sequences as described above, the multimer can suitably be devoid of linkers. The linkers may e.g. comprise or consist essentially of a peptide sequence defined by, or having at least 90% identity or at least 95% identity, with an amino acid sequence selected from the group consisting of APKVDAKFDKE, APKVDNKFNKE, APKADNKFNKE, APKVFDKE, APAKFDKE, AKFDKE, APKVDA, VDAKFDKE, APKKFDKE, APK, APKYEDGVDAKFDKE and YEDG or alternatively selected from the group consisting of APKVDAKFDKE, APKVFDKE, APAKFDKE, AKFDKE, APKVDA, VDAKFDKE, APKKFDKE, APKYEDGVDAKFDKE and YEDG. They can also consist essentially of a peptide sequence defined by or having at least 90% identity or at least 95% identity with an amino acid sequence selected from the group consisting of APKADNKFNKE, APKVFDKE, APAKFDKE, AKFDKE, APKVDA, VDAKFDKE, APKKFDKE, APK and APKYEDGVDAKFDKE. The nature of such a linker should preferably not destabilize the spatial conformation of the protein units. This can e.g. be achieved by avoiding the presence of proline in the linkers. Furthermore, said linker should preferably also be sufficiently stable in alkaline environments not to impair the properties of the mutated protein units. For this purpose, it is advantageous if the linkers do not contain asparagine. It can additionally be advantageous if the linkers do not contain glutamine. Suitable linker sequences are further described in U.S. Pat. No. 10,703,74, hereby incorporated by reference in its entirety.

The multimer may further at the N-terminal end comprise a plurality of amino acid residues e.g. originating from the cloning process or constituting a residue from a cleaved off signaling sequence, herein called an N-terminal sequence. The number of additional amino acid residues may e.g. be 20 or less, such as 15 or less, such as 10 or less or 5 or less. As a specific example, the multimer may comprise an AQ, AQGT, VDAKFDKE, AQVDAKFDKE, AQGTVDAKFDKE, AQYEDGKQYTDT, AQYEDGKQYT, AQKDQTWYTG, AQHDEAQQEA, AQGGGSGGGS, AQYEDGKQYGT, AQYEDGKQGT, AQYEDGKQYTTLEKGT, AQYEDGKQYTTLEKPVAGGT, AQYEDGKQYTET, AQYEDGKQYTDT, AQYEDGKQYTAT, AQYEDGKQYEDT, AQHHHHHHHHGT, AQHHHHHHGT or AQHDEAQQEAGT sequence at the N-terminal end. N-terminal sequences are further discussed in US20200318120, hereby incorporated by reference in its entirety.

In some embodiments, the polypeptide and/or multimer, as disclosed above, further comprises at the C-terminal or N-terminal end one or more coupling elements, selected from the group consisting of one or more cysteine residues, a plurality of lysine residues and a plurality of histidine residues. The coupling element(s) may also be located within 1-5 amino acid residues, such as within 1-3 or 1-2 amino acid residues from the C-terminal or N-terminal end. The coupling element may e.g. be a single cysteine at the C-terminal end. The coupling element(s) may be directly linked to the C- or N-terminal end, or it/they may be linked via a stretch comprising up to 15 amino acids, such as 1-5, 1-10 or 5-10 amino acids. This stretch should preferably also be sufficiently stable in alkaline environments not to impair the properties of the mutated protein. For this purpose, it is advantageous if the stretch does not contain asparagine. It can additionally be advantageous if the stretch does not contain glutamine. An advantage of having a C-terminal cysteine is that endpoint coupling of the protein can be achieved through reaction of the cysteine thiol with an electrophilic group on a support. This provides excellent mobility of the coupled protein which is important for the binding capacity.

In accordance with the description above, the multimer may e.g. have a structure:

[N-terminal sequence]-([Polypeptide]ₙ-[)Coupling element], or
[N-terminal sequence]-([Polypeptide]-[Linker](ₙ-[Coupling element],
where N-terminal sequence, Polypeptide, Linker and Coupling element are as discussed above and where n is 2-10, as exemplified by 2, 3, 4, 5, 6, 7, 8, 9 or 10, such as 4, 5, 6 or 7.

The alkali stability of the polypeptide or multimer can be assessed by coupling it to an SPR chip, e.g. to Biacore CM5 sensor chips as described in the examples, using e.g. NHS- or maleimide coupling chemistries, and measuring the immunoglobulin-binding capacity of the chip, typically using polyclonal human IgG, before and after incubation in alkaline solutions at a specified temperature, e.g. 22 +/−2° C. The incubation can e.g. be performed in 0.5 M NaOH for a number of 10 min cycles, such as 100, 200 or 300 cycles. The IgG capacity of the matrix after 100 10 min incubation cycles in 0.5 M NaOH at 22 +/−2° C. can be at least 55, such as at least at least 80 or at least 90% of the IgG capacity before the incubation. Alternatively, the remaining IgG capacity after 100 cycles for a particular mutant measured as above can be compared with the remaining IgG capacity for the parental polypeptide/multimer. In this case, the remaining IgG capacity for the mutant may be at least 105%, such as at least 110%, at least 125%, at least 150% or at least 200% of the parental polypeptide/multimer.

In a third aspect the present invention discloses a nucleic acid encoding a polypeptide or multimer according to any embodiments disclosed above. Thus, the invention encompasses all forms of the present nucleic acid sequence such as the RNA and the DNA encoding the polypeptide or multimer. The invention embraces a vector, such as a plasmid, which in addition to the coding sequence comprises the required signal sequences for expression of the polypeptide or multimer according the invention. In some embodiments, the vector comprises nucleic acid encoding a multimer according to the invention, wherein the separate nucleic acids encoding each unit may have homologous or heterologous DNA sequences.

In a fourth aspect the present invention discloses an expression system, which comprises a nucleic acid or a vector as disclosed above. The expression system may e.g. be a gram-positive or gram-negative prokaryotic host cell system, e.g. E.coli or Bacillus sp. which has been modified to express the present polypeptide or multimer. In alternative embodiments, the expression system is a eukaryotic host cell system, such as a yeast, e.g. Pichia pastoris or Saccharomyces cerevisiae, or mammalian cells, e.g. CHO cells.

In a fifth aspect, the present invention discloses a separation matrix, wherein a plurality of polypeptides or multimers, denoted Fc-binding ligands, according to any embodiments disclosed above have been coupled to a solid support. The separation matrix may comprise at least 11, such as 11-25, 15-25 or 15-22 mg/ml Fc-binding ligands covalently coupled to a porous support, wherein suitably:

a) the ligands comprise multimers or polypeptides as discussed above,
b) the porous support comprises cross-linked polymer particles having a volume-weighted median diameter (d50,v) of 50-75, such as 56-70 or 56-66 micrometers and a dry solids weight of 55-80, such as 60-78 or 65-78, mg/ml. The cross-linked polymer particles may further have a pore size corresponding to an inverse gel filtration chromatography Kd value of 0.69-0.85, such as 0.70-0.85 or 0.69-0.80, for dextran of Mw 110 kDa. Suitably, the cross-linked polymer particles can have a high rigidity, to be able to withstand high flow rates. The rigidity can be measured with a pressure-flow test, where a column packed with the matrix is subjected to increasing flow rates of distilled water. The pressure is increased stepwise and the flow rate and back pressure measured, until the flow rate starts to decrease with increasing pressures. The maximum flow rate achieved and the maximum pressure (the back pressure corresponding to the maximum flow rate) are measured and used as measures of the rigidity. When measured in a FineLine™ 35 column (Cytiva) at a bed height of 300 +/−10 mm, the max pressure can suitably be at least 0.58 MPa, such as at least 0.60 MPa. This allows for the use of smaller particle diameters, which is beneficial for the dynamic capacity. The multimers may e.g. comprise tetramers, pentamers, hexamers or heptamers of alkali-stabilized Protein A domains, such as hexamers of alkali-stabilized Protein A domains. The combination of the high ligand contents with the particle size range, the dry solids weight range and the optional Kd range provides for a high binding capacity, e.g. such that the 10% breakthrough dynamic binding capacity for IgG is at least 45 mg/ml, such as at least 50 or at least 55 mg/ml at 2.4 min residence time.

Alternatively, or additionally, the 10% breakthrough dynamic binding capacity for IgG may be at least 60 mg/ml, such as at least 65, at least 70 or at least 75 mg/ml at 6 min residence time. The alkali-stabilized Protein A multimers are highly selective for IgG and the separation matrix can suitably have a dissociation constant for human IgG2 of below 0.2 mg/ml, such as below 0.1 mg/ml, in 20 mM phosphate buffer, 180 mM NaCl, pH 7.5. This can be determined according to the adsorption isotherm method described in N Pakiman et al: J Appl Sci 12, 1136-1141 (2012).

In certain embodiments the invention discloses a separation matrix comprising at least 15, such as 15-21 or 15-18 mg/ml Fc-binding ligands covalently coupled to a porous support, wherein the ligands comprise multimers of alkali-stabilized Protein A domains. These multimers can suitably be as disclosed in any of the embodiments described above or as specified below.

Such a matrix is useful for separation of immunoglobulins or other Fc-containing proteins and, due to the improved alkali stability of the polypeptides/multimers, the matrix will withstand highly alkaline conditions during cleaning, which is essential for long-term repeated use in a bioprocess separation setting. The alkali stability of the matrix can be assessed by measuring the immunoglobulin-binding capacity, typically using polyclonal human IgG, before and after incubation in alkaline solutions at a specified temperature, e.g. 22 +/−2° C. The incubation can e.g. be performed in 0.5 M or 1.0 M NaOH for a number of 15 min cycles, such as 100, 200 or 300 cycles, corresponding to a total incubation time of 25, 50 or 75 h. The IgG capacity of the matrix after 96-100 15 min incubation cycles or a total incubation time of 24 or 25 h in 0.5 M NaOH at 22 +/−2° C. can be at least 80, such as at least 85, at least 90 or at least 95% of the IgG capacity before the incubation. The capacity of the matrix after a total incubation time of 24 h in 1.0 M NaOH at 22 +/−2° C. can be at least 70, such as at least 80 or at least 90% of the IgG capacity before the incubation. The the 10% breakthrough dynamic binding capacity (Qb10%) for IgG at 2.4 min or 6 min residence time may e.g. be reduced by less than 20% after incubation 31 h in 1.0 M aqueous NaOH at 22 +/−2 C.

As the skilled person will understand, the expressed polypeptide or multimer should be purified to an appropriate extent before being immobilized to a support. Such purification methods are well known in the field, and the immobilization of protein-based ligands to supports is easily carried out using standard methods. Suitable methods and supports will be discussed below in more detail.

The solid support of the matrix according to the invention can be of any suitable well-known kind. A conventional affinity separation matrix is often of organic nature and based on polymers that expose a hydrophilic surface to the aqueous media used, i.e. expose hydroxy (—OH), carboxy (—COOH), carboxamido (—CONH₂, possibly in N-substituted forms), amino (—NH₂, possibly in substituted form), oligo- or polyethylenoxy groups on their external and, if present, also on internal surfaces. The solid support can suitably be porous. The porosity can be expressed as a Kay or Kd value (the fraction of the pore volume available to a probe molecule of a particular size) measured by inverse size exclusion chromatography, e.g. according to the methods described in Gel Filtration Principles and Methods, Pharmacia LKB Biotechnology 1991, pp 6-13. Kay is determined as the ratio (Vₑ−V₀)/(Vₜ−V₀), where Ve is the elution volume of a probe molecule (e.g. Dextran 110 kD), Vo is the void volume of the column (e.g. the elution volume of a high Mw void marker, such as raw dextran) and Vₜ is the total volume of the column. Kd can be determined as (Vₑ−V0)/Vₜ, where Vₜ is the elution volume of a salt (e.g. NaCl) able to access all the volume except the matrix volume (the volume occupied by the matrix polymer molecules). By definition, both Kd and Kay values always lie within the range 0 − 1. The Kay value can advantageously be 0.6-0.95, e.g. 0.7-0.90 or 0.6-0.8, as measured with dextran of Mw 110 kDa as a probe molecule. The Kd value as measured with dextran of Mw 110 kDa can suitably be 0.68-0.90, such as 0.68-0.85 or 0.70-0.85. An advantage of this is that the support has a large fraction of pores able to accommodate both the polypeptides/multimers of the invention and immunoglobulins binding to the polypeptides/multimers and to provide mass transport of the immunoglobulins to and from the binding sites.

The polypeptides or multimers may be attached to the support via conventional coupling techniques utilising e.g. thiol, amino and/or carboxy groups present in the ligand. Bisepoxides, epichlorohydrin, CNBr, N-hydroxysuccinimide (NHS) etc. are well-known coupling reagents. Between the support and the polypeptide/multimer, a molecule known as a spacer can be introduced, which improves the availability of the polypeptide/multimer and facilitates the chemical coupling of the polypeptide/multimer to the support. Depending on the nature of the polypeptide/multimer and the coupling conditions, the coupling may be a multipoint coupling (e.g. via a plurality of lysines) or a single point coupling (e.g. via a single cysteine). Alternatively, the polypeptide/multimer may be attached to the support by non-covalent bonding, such as physical adsorption or biospecific adsorption.

In some embodiments the matrix comprises 5-25, such as 5-20 mg/ml, 5-15 mg/ml, 5-11 mg/ml or 6-11 mg/ml of the polypeptide or multimer coupled to the support.

The amount of coupled polypeptide/multimer can be controlled by the concentration of polypeptide/multimer used in the coupling process, by the activation and coupling conditions used and/or by the pore structure of the support used. As a general rule the absolute binding capacity of the matrix increases with the amount of coupled polypeptide/multimer, at least up to a point where the pores become significantly constricted by the coupled polypeptide/multimer. Without being bound by theory, it appears though that for the Kd values recited for the support, the constriction of the pores by coupled ligand is of lower significance. The relative binding capacity per mg coupled polypeptide/multimer will decrease at high coupling levels, resulting in a cost-benefit optimum within the ranges specified above.

In certain embodiments the polypeptides or multimers are coupled to the support via thioether bonds. Methods for performing such coupling are well-known in this field and easily performed by the skilled person in this field using standard techniques and equipment. Thioether bonds are flexible and stable and generally suited for use in affinity chromatography.

In particular when the thioether bond is via a terminal or near-terminal cysteine residue on the polypeptide or multimer, the mobility of the coupled polypeptide/multimer is enhanced which provides improved binding capacity and binding kinetics. In some embodiments the polypeptide/multimer is coupled via a C-terminal cysteine provided on the protein as described above. This allows for efficient coupling of the cysteine thiol to electrophilic groups, e.g. epoxide groups, halohydrin groups etc. on a support, resulting in a thioether bridge coupling.

In certain embodiments the support comprises a polyhydroxy polymer, such as a polysaccharide. Examples of polysaccharides include e.g. dextran, starch, cellulose, pullulan, agar, agarose etc. Polysaccharides are inherently hydrophilic with low degrees of nonspecific interactions, they provide a high content of reactive (activatable) hydroxyl groups and they are generally stable towards alkaline cleaning solutions used in bioprocessing.

In some embodiments the support comprises agar or agarose. The supports used in the present invention can easily be prepared according to standard methods, such as inverse suspension gelation (S Hjertén: Biochim Biophys Acta 79(2), 393-398 (1964). Alternatively, the base matrices are commercially available products, such as crosslinked agarose beads sold under the name of SEPHAROSE™ FF (Cytiva). In an embodiment, which is especially advantageous for large-scale separations, the support has been adapted to increase its rigidity using the methods described in U.S. Pat. No. 6,602,990 or U.S. Pat. No. 7,396,467, which are hereby incorporated by reference in their entireties, and hence renders the matrix more suitable for high flow rates.

In certain embodiments the support, such as a polymer, polysaccharide or agarose support, is crosslinked, such as with hydroxyalkyl ether crosslinks. Crosslinker reagents producing such crosslinks can be e.g. epihalohydrins like epichlorohydrin, diepoxides like butanediol diglycidyl ether, allylating reagents like allyl halides or allyl glycidyl ether. Crosslinking is beneficial for the rigidity of the support and improves the chemical stability. Hydroxyalkyl ether crosslinks are alkali stable and do not cause significant nonspecific adsorption.

Alternatively, the solid support is based on synthetic polymers, such as polyvinyl alcohol, polyhydroxyalkyl acrylates, polyhydroxyalkyl methacrylates, polyacrylamides, polymethacrylamides etc. In case of hydrophobic polymers, such as matrices based on divinyl and monovinyl-substituted benzenes, the surface of the matrix is often hydrophilised to expose hydrophilic groups as defined above to a surrounding aqueous liquid. Such polymers are easily produced according to standard methods, see e.g. “Styrene based polymer supports developed by suspension polymerization” (R Arshady: Chimica e L'Industria 70(9), 70-75 (1988)). Alternatively, a commercially available product, such as SOURCE™ (Cytiva) is used. In another alternative, the solid support according to the invention comprises a support of inorganic nature, e.g. silica, zirconium oxide etc.

In yet further embodiments, the solid support is in another form such as a surface, a chip, capillaries, or a filter (e.g. a membrane or a depth filter matrix). When the support is a membrane, the membrane can suitably be a fibrous membrane comprising nanofibers of 10-1000 nm diameter, as described in U.S. Pat. No. 10,696,714, U.S. Pat. No. 10,850,259 or U.S. Pat. No. 16/959,373, hereby incorporated by reference in their entireties. The nanofibers can suitably be cellulose nanofibers.

As regards the shape of the matrix according to the invention, in certain embodiments the matrix is in the form of a porous monolith. In alternative embodiments, the matrix is in beaded or particle form that can be porous or non-porous. Matrices in beaded or particle form can be used as a packed bed or in a suspended form. Suspended forms include those known as expanded beds and pure suspensions, in which the particles or beads are free to move. In case of monoliths, packed bed and expanded beds, the separation procedure commonly follows conventional chromatography with a concentration gradient. In case of pure suspension, batch-wise mode will be used.

In a sixth aspect, the present invention discloses a method of isolating an immunoglobulin, wherein a separation matrix as disclosed above is used. The method may comprise the steps of:

a) contacting a liquid sample comprising an immunoglobulin with a separation matrix as disclosed above,
b) washing the separation matrix with a washing liquid,
c) eluting the immunoglobulin from the separation matrix with an elution liquid, and
d) cleaning the separation matrix with a cleaning liquid, which may comprise 0.1-1.0 M NaOH or KOH, such as 0.4-1.0 M NaOH or KOH.
Steps a) — d) may be repeated at least 10 times, such as at least 50 times, 50-200, 50-300 or 50-400 times.

In certain embodiments, the method comprises the steps of:

a) contacting a liquid sample comprising an immunoglobulin with a separation matrix as disclosed above,
b) washing said separation matrix with a washing liquid,
c) eluting the immunoglobulin from the separation matrix with an elution liquid, and
d) cleaning the separation matrix with a cleaning liquid, which can alternatively be called a cleaning-in-place (CIP) liquid, e.g. with a contact (incubation) time of at least 10 min.
The method may also comprise steps of, before step a), providing an affinity separation matrix according to any of the embodiments described above and providing a solution comprising an immunoglobulin and at least one other substance as a liquid sample and of, after step c), recovering the eluate and optionally subjecting the eluate to further separation steps, e.g. by anion or cation exchange chromatography, multimodal chromatography and/or hydrophobic interaction chromatography. Suitable compositions of the liquid sample, the washing liquid and the elution liquid, as well as the general conditions for performing the separation are well known in the art of affinity chromatography and in particular in the art of Protein A chromatography. The liquid sample comprising an Fc-containing protein and at least one other substance may comprise host cell proteins (HCP), such as CHO cell, E Coli or yeast proteins. Contents of CHO cell and E Coli proteins can conveniently be determined by immunoassays directed towards these proteins, e.g. the CHO HCP or E Coli HCP ELISA kits from Cygnus Technologies. The host cell proteins or CHO cell/E Coli proteins may be desorbed during step b).

The elution may be performed by using any suitable solution used for elution from Protein A media. This can e.g. be a solution or buffer with pH 5 or lower, such as pH 2.5-5 or 3-5. It can also in some cases be a solution or buffer with pH 11 or higher, such as pH 11-14 or pH 11-13. In some embodiments the elution buffer or the elution buffer gradient comprises at least one mono- di- or trifunctional carboxylic acid or salt of such a carboxylic acid. In certain embodiments the elution buffer or the elution buffer gradient comprises at least one anion species selected from the group consisting of acetate, citrate, glycine, succinate, phosphate, and formiate.

In some embodiments, the cleaning liquid is alkaline, such as with a pH of 13-14. Such solutions provide efficient cleaning of the matrix, in particular at the upper end of the interval

In certain embodiments, the cleaning liquid comprises 0.1-2.0 M NaOH or KOH, such as 0.5-2.0 or 0.5-1.0 M NaOH or KOH. These are efficient cleaning solutions, and in particular so when the NaOH or KOH concentration is above 0.1 M or at least 0.5 M. The high stability of the polypeptides of the invention enables the use of such strongly alkaline solutions.

The method may also include a step of sanitizing the matrix with a sanitization liquid, which may e.g. comprise a peroxide, such as hydrogen peroxide and/or a peracid, such as peracetic acid or performic acid.

In some embodiments, steps a)-d) are repeated at least 10 times, such as at least times, 50-200, 50-300 or 50-500 times. This is important for the process economy in that the matrix can be re-used many times.

Steps a)-c) can also be repeated at least 10 times, such as at least 50 times, 50-200, 50-300 or 50-500 times, with step d) being performed after a plurality of instances of step c), such that step d) is performed at least 10 times, such as at least 50 times. Step d) can e.g. be performed every second to twentieth instance of step c).

### EXAMPLES

### Mutagenesis of Protein

Site-directed mutagenesis was performed by a two-step PCR using oligonucleotides coding for the mutations. As template a plasmid containing a single domain of either Z, B or C was used. The PCR fragments were ligated into an E. coli expression vector. DNA sequencing was used to verify the correct sequence of inserted fragments. To form multimers of mutants an Acc I site located in the starting codons (GTA GAC) of the B, C or Z domain was used, corresponding to amino acids VD. The vector for the monomeric domain was digested with Acc I and phosphatase treated. Acc I sticky-ends primers were designed, specific for each variant, and two overlapping PCR products were generated from each template. The PCR products were purified and the concentration was estimated by comparing the PCR products on a 2% agarose gel. Equal amounts of the pair wise PCR products were hybridized (90° C. ->25° C. in 45 min) in ligation buffer. The resulting product consists approximately to ¼ of fragments likely to be ligated into an Acc I site (correct PCR fragments and/or the digested vector). After ligation and transformation colonies were PCR screened to identify constructs containing the desired mutant. Positive clones were verified by DNA sequencing.

### Construct Expression and Purification

The constructs were expressed in the bacterial periplasm by fermentation of E. coli K12 in standard media. After fermentation the cells were heat-treated to release the periplasm content into the media. The constructs released into the medium were recovered by microfiltration with a membrane having a 0.2 μm pore size.

Each construct, now in the permeate from the filtration step, was purified by affinity. The permeate was loaded onto a chromatography medium containing immobilized IgG (IgG Sepharose 6FF, Cytiva). The loaded product was washed with phosphate buffered saline and eluted by lowering the pH.

The elution pool was adjusted to a neutral pH (pH 8) and reduced by addition of dithiothreitol. The sample was then loaded onto an anion exchanger. After a wash step the construct was eluted in a NaCl gradient to separate it from any contaminants. The elution pool was concentrated by ultrafiltration to 40-50 mg/ml. It should be noted that the successful affinity purification of a construct on an immobilized IgG medium indicates that the construct in question has a high affinity to IgG.

The purified ligands were analyzed with RPC LC-MS to determine the purity and to ascertain that the molecular weight corresponded to the expected (based on the amino acid sequence).

### Example 1

The purified monomeric ligands listed in Table 1, further comprising an AQYEDGKQYTDTVDAKFDKE leader sequence at the N-terminus and an APK tail sequence at the C-terminus, were immobilized on Biacore CM5 sensor chips (Cytiva, Sweden), using the amine coupling kit of Cytiva (for carbodiimide coupling of amines on the carboxymethyl groups on the chip) in an amount sufficient to give a signal strength of about 200-1500 RU in a Biacore surface plasmon resonance (SPR) instrument (Cytiva, Sweden) . To follow the IgG binding capacity of the immobilized surface lmg/ml human polyclonal IgG (Gammanorm) was flowed over the chip and the signal strength (proportional to the amount of binding) was noted. The surface was then cleaned-in-place (CIP), i.e. flushed with 500 mM NaOH for 10 minutes at room temperature (22 +/−2° C.). This was repeated for 96-100 cycles and the immobilized ligand alkaline stability was followed as the remaining IgG binding capacity (signal strength) after each cycle. The ligand Zvar(Q9A,N11E,Q40V,A42K,N43A,L44Di (SEQ ID NO: 12), as disclosed in U.S. Pat. No. 10,703,774, with an AQYEDGKQYTDT leader sequence was used as a reference (note that SEQ ID NO:12 includes VDAKFDKE at the N-terminal end and APK at the C-terminal end). The results are shown in Table 1 and indicate that both SEQ ID NO: 8 and SEQ ID NO: 9 are significantly more alkali-stable than the reference.

TABLE 1
Monomeric ligands, evaluated by Biacore (0.5M NaOH).

| | | | Reference | |
|---|---|---|---|---|
| | | Capacity | capacity | Capacity |
| | | after 100 | after 100 | relative |
| Ligand | Sequence | cycles | cycles | to Ref |
| Zvar(Q9A, N11R, Q40V, | SEQ ID | 74% | 65% | 1.14 |
| A42K, N43A, L44I)1 | NO 8 | | | |
| Zvar(Q9W, N11E, Q40V, | SEQ ID | 70% | 64% | 1.09 |
| A42K, N43A, L44I)1 | NO 9 | | | |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties, as if they were individually incorporated.

1. An Fc-binding polypeptide comprising an amino acid sequence as defined by, or having at least 95%, such as at least 98% identity, to SEQ ID NO: 8 or SEQ ID NO: 9.

2. An Fc-binding polypeptide comprising a sequence as defined by, or having at least 98% identity to, SEQ ID NO 11:
(SEQ ID NO 11)
X₁Q X₂AFYEILHLP NLTEEQRNAF IQSLKDDPSX₃ SKAILAEAKK
LNDAQ
wherein individually of each other:
X1=A or W
X2=E or R
X3=V or Q,
with the proviso that when X1 is A, X2=R and when X2=E, X1=W.

3. The Fc-binding polypeptide of claim 2, wherein X3=V.

4. The Fc-binding polypeptide of claim 1, further comprising a 1-20 amino acid leader sequence.

5. The Fc-binding polypeptide of claim 4, wherein the leader sequence is defined by or has at least 80% identity, such as at least 90% identity or at least 95% identity, with an amino acid sequence selected from the group consisting of VFDKE, AKFDKE, VDA, VDAKFDKE, KFDKE, KVDKE, KADKE, ADNKFNKE, VDNKFNKE, YEDGVDAKFDKE, AQYEDGKQYTDT and AQYEDGKQYTDTVDAKFDKE.

6. The Fc-binding polypeptide of claim 4, wherein the leader sequence is defined by or has at least 80% identity, such as at least 90% identity or at least 95% identity, with an amino acid sequence selected from the group consisting of VFDKE, AKFDKE, VDA, VDAKFDKE, KFDKE, KVDKE, KADKE, YEDGVDAKFDKE and AQYEDGKQYTDT and AQYEDGKQYTDTVDAKFDKE.

7. The Fc-binding polypeptide of claim 1, further comprising a 1-5 amino acid tail sequence. , further

8. The Fc-binding polypeptide of claim 7, wherein the tail sequence is defined by or has at least 60% identity with an amino acid sequence selected from the group consisting of AP, APK and APA.

9. A multimer comprising a plurality of linked Fc-binding polypeptides according to claim 1.

10. The multimer of claim 9, which is a dimer, trimer, tetramer, pentamer, hexamer or heptamer.

11. The multimer of claim 9--E*-4, wherein the polypeptides are linked by linkers comprising up to 25 amino acids, such as 3-25 or 3-20 amino acids.

12. The multimer of claim 9, wherein at least two polypeptides are linked by linkers comprising a sequence having at least 90% identity with an amino acid sequence selected from the group consisting of APKVDAKFDKE, APKVDNKFNKE, APKADNKFNKE, APKVFDKE, APAKFDKE, AKFDKE, APKVDA, VDAKFDKE, APKKFDKE, APK, APKYEDGVDAKFDKE and YEDG.

13. The multimer of claim 9, wherein at least two polypeptides are linked by linkers comprising a sequence having at least 90% identity with an amino acid sequence selected from the group consisting of APKVDAKFDKE, APKVFDKE, APAKFDKE, AKFDKE, APKVDA, VDAKFDKE, APKKFDKE, APK, APKYEDGVDAKFDKE and YEDG.

14. The multimer of claim 9, further comprising an N-terminal sequence of 1-20 amino acid residues at the N-terminal end.

15. The multimer of claim 14, wherein said N-terminal sequence comprises a sequence selected from the group consisting of AQ, AQGT, VDAKFDKE, AQVDAKFDKE, AQGTVDAKFDKE, AQYEDGKQYTDT, AQYEDGKQYT, AQKDQTWYTG, AQHDEAQQEA, AQGGGSGGGS, AQYEDGKQYGT, AQYEDGKQGT, AQYEDGKQYTTLEKGT, AQYEDGKQYTTLEKPVAGGT, AQYEDGKQYTET, AQYEDGKQYTDT, AQYEDGKQYTAT, AQYEDGKQYEDT, AQHHHHHHHHGT, AQHHHHHHGT and AQHDEAQQEAGT.

16. The multimer of claim 9, further comprising at the C-terminal or N-terminal end one or more coupling elements, selected from the group consisting of one or more cysteine residues, a plurality of lysine residues and a plurality of histidine residues.

17. The multimer of claim 16, comprising a single cysteine residue at the C-terminus or within 5 amino acid residues from the C-terminus.

18. A nucleic acid or vector encoding the polypeptide or multimer according to claim 1.

19. An expression system comprising the nucleic acid or a vector of claim 18.

20. A separation matrix comprising a plurality of polypeptides or multimers according to claim 1, covalently coupled to a solid support.

21. The separation matrix of claim 20, comprising at least 11 mg of the polypeptides or multimers per ml of a porous support.

22. The separation matrix of claim 20, wherein said support comprises porous polymer particles.

23. The separation matrix of claim 22, wherein said porous polymer particles are crosslinked agarose particles.

24. The separation matrix of claim 20, wherein said support comprises a porous membrane.

25. The separation matrix of claim 24, wherein said support comprises a fibrous membrane comprising nanofibers of 10-1000 nm diameter.

26. The separation matrix of claim 25, wherein said nanofibers are cellulose nanofibers.

27. A method of isolating an immunoglobulin, comprising the steps of:
a) contacting a liquid sample comprising an immunoglobulin with a separation matrix according to claim 1,
b) washing said separation matrix with a washing liquid,
c) eluting the immunoglobulin from the separation matrix with an elution liquid, and
d) cleaning the separation matrix with a cleaning liquid.

28. The method of claim 27, wherein the cleaning liquid comprises 0.1-1.0 M NaOH or KOH, such as 0.4-1.0 M NaOH or KOH.

29. The method of claim 27, wherein steps a)-d) are repeated at least 10 times, such as at least 50 times, 50-200 or 50-400 times.


SEQUENCE LISTING (54 sequences)

SEQ ID NO: 1 - protein, 51 aa, Staphylococcus aureus
AQQNAFYQVLNMPNLNADQRNGFIQSLKDDPSQSANVLGEAQKLNDSQAPK

SEQ ID NO: 2 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 3 - protein, 58 aa, Staphylococcus aureus
ADNNFNKEQQNAFYEILNMPNLNEEQRNGFIQSLKDDPSQSANLLAEAKKLNESQAPK

SEQ ID NO: 4 - protein, 58 aa, Staphylococcus aureus [sequence omitted]

SEQ ID NO: 5 - protein, 58 aa, Staphylococcus aureus
ADNKFNKEQQNAFYEILHLPNLTEEQRNGFIQSLKDDPSVSKEILAEAKKLNDAQAPK

SEQ ID NO: 6 - protein, 58 aa, Artificial Sequence [sequence omitted]

SEQ ID NO: 7 - protein, 58 aa, Artificial Sequence [sequence omitted]

SEQ ID NO: 8 - protein, 47 aa, Artificial Sequence
  Zvar (Q9A,N11R,Q40V,A42K,N43A,L44I)
AQRAFYEILHLPNLTEEQRNAFIQSLKDDPSVSKAILAEAKKLNDAQ

SEQ ID NO: 9 - protein, 47 aa, Artificial Sequence
  Zvar (Q9W,N11E,Q40V,A42K,N43A,L44I)
WQEAFYEILHLPNLTEEQRNAFIQSLKDDPSVSKAILAEAKKLNDAQ

SEQ ID NO: 10 - protein, 47 aa, Artificial Sequence
  Zvar (Q9W,N11R,Q40V,A42K,N43A,L44I)
WQRAFYEILHLPNLTEEQRNAFIQSLKDDPSVSKAILAEAKKLNDAQ

SEQ ID NO: 11 - protein, 47 aa, Artificial Sequence
  Zvar variant
  misc_feature (1)..(1): Xaa can be any naturally occurring amino acid
  misc_feature (3)..(3): Xaa can be any naturally occurring amino acid
  misc_feature (32)..(32): Xaa can be any naturally occurring amino acid
XQXAFYEILHLPNLTEEQRNAFIQSLKDDPSXSKAILAEAKKLNDAQ

SEQ ID NO: 12 - protein, 58 aa, Artificial Sequence
  Zvar (Q9A,N11E,Q40V,A42K,N43A,L44I)
VDAKFDKEAQEAFYEILHLPNLTEEQRNAFIQSLKDDPSVSKAILAEAKKLNDAQAPK

SEQ ID NO: 13 - protein, 5 aa, Artificial Sequence
  Leader
VFDKE

SEQ ID NO: 14 - protein, 6 aa, Artificial Sequence
  Leader
AKFDKE

SEQ ID NO: 15 - protein, 8 aa, Artificial Sequence
  Leader
VDAKFDKE

SEQ ID NO: 16 - protein, 5 aa, Artificial Sequence
  Leader
KFDKE

SEQ ID NO: 17 - protein, 5 aa, Artificial Sequence
  Leader
KVDKE

SEQ ID NO: 18 - protein, 5 aa, Artificial Sequence
  Leader
KADKE

SEQ ID NO: 19 - protein, 12 aa, Artificial Sequence
  Leader
YEDGVDAKFDKE

SEQ ID NO: 20 - protein, 12 aa, Artificial Sequence
  Leader
AQYEDGKQYTDT

SEQ ID NO: 21 - protein, 20 aa, Artificial Sequence
  Leader
AQYEDGKQYTDTVDAKFDKE

SEQ ID NO: 22 - protein, 8 aa, Artificial Sequence
  Leader
ADNKFNKE

SEQ ID NO: 23 - protein, 8 aa, Artificial Sequence
  Leader
VDNKFNKE

SEQ ID NO: 24 - protein, 11 aa, Artificial Sequence
  Linker
APKVDAKFDKE

SEQ ID NO: 25 - protein, 11 aa, Artificial Sequence
  Linker
APKVDNKFNKE

SEQ ID NO: 26 - protein, 11 aa, Artificial Sequence
  Linker
APKADNKFNKE

SEQ ID NO: 27 - protein, 8 aa, Artificial Sequence
  Linker
APKVFDKE

SEQ ID NO: 28 - protein, 8 aa, Artificial Sequence
  Linker
APAKFDKE

SEQ ID NO: 29 - protein, 6 aa, Artificial Sequence
  Linker
AKFDKE

SEQ ID NO: 30 - protein, 6 aa, Artificial Sequence
  Linker
APKVDA

SEQ ID NO: 31 - protein, 8 aa, Artificial Sequence
  Linker
VDAKFDKE

SEQ ID NO: 32 - protein, 8 aa, Artificial Sequence
  Linker
APKKFDKE

SEQ ID NO: 33 - protein, 15 aa, Artificial Sequence
  Linker
APKYEDGVDAKFDKE

SEQ ID NO: 34 - protein, 4 aa, Artificial Sequence
  Linker
YEDG

SEQ ID NO: 35 - protein, 4 aa, Artificial Sequence
  N-terminal sequence
AQGT

SEQ ID NO: 36 - protein, 8 aa, Artificial Sequence
  N-terminal sequence
VDAKFDKE

SEQ ID NO: 37 - protein, 10 aa, Artificial Sequence
  N-terminal sequence
AQVDAKFDKE

SEQ ID NO: 38 - protein, 12 aa, Artificial Sequence
  N-terminal sequence
AQGTVDAKFDKE

SEQ ID NO: 39 - protein, 12 aa, Artificial Sequence
  N-terminal sequence
AQYEDGKQYTDT

SEQ ID NO: 40 - protein, 10 aa, Artificial Sequence
  N-terminal sequence
AQYEDGKQYT

SEQ ID NO: 41 - protein, 10 aa, Artificial Sequence
  N-terminal sequence
AQKDQTWYTG

SEQ ID NO: 42 - protein, 10 aa, Artificial Sequence
  N-terminal sequence
AQHDEAQQEA

SEQ ID NO: 43 - protein, 10 aa, Artificial Sequence
  N-terminal sequence
AQGGGSGGGS

SEQ ID NO: 44 - protein, 11 aa, Artificial Sequence
  N-terminal sequence
AQYEDGKQYGT

SEQ ID NO: 45 - protein, 10 aa, Artificial Sequence
  N-terminal sequence
AQYEDGKQGT

SEQ ID NO: 46 - protein, 16 aa, Artificial Sequence
  N-terminal sequence
AQYEDGKQYTTLEKGT

SEQ ID NO: 47 - protein, 20 aa, Artificial Sequence
  N-terminal sequence
AQYEDGKQYTTLEKPVAGGT

SEQ ID NO: 48 - protein, 12 aa, Artificial Sequence
  N-terminal sequence
AQYEDGKQYTET

SEQ ID NO: 49 - protein, 12 aa, Artificial Sequence
  N-terminal sequence
AQYEDGKQYTDT

SEQ ID NO: 50 - protein, 12 aa, Artificial Sequence
  N-terminal sequence
AQYEDGKQYTAT

SEQ ID NO: 51 - protein, 12 aa, Artificial Sequence
  N-terminal sequence
AQYEDGKQYEDT

SEQ ID NO: 52 - protein, 12 aa, Artificial Sequence
  N-terminal sequence
AQHHHHHHHHGT

SEQ ID NO: 53 - protein, 10 aa, Artificial Sequence
  N-terminal sequence
AQHHHHHHGT

SEQ ID NO: 54 - protein, 12 aa, Artificial Sequence
  N-terminal sequence
AQHDEAQQEAGT